(19) United States
(12) Patent Application Publication
(10) Pub. No.: US 2015/0005853 A1
(43) Pub. Date: Jan. 1, 2015

(54) PHOTOTHERAPY DEVICE

(71) Applicant: AVATAR-STAR TECHNOLOGY CORP., TAIPEI CITY (TW)

(72) Inventor: HSIU-CHIH CHANG, TAIPEI CITY (TW)

(21) Appl. No.: 13/930,591

(22) Filed: Jun. 28, 2013

(51) Int. Cl. A61N 5/06

(52) U.S. Cl. CPC A61N 5/0616
USPC 607/90

(57) ABSTRACT

A phototherapy device comprises includes a translucent insulating carrier, and an illuminant module disposed inside the translucent insulating carrier, where in the illuminant module comprises a circuit board, a power supply unit, a switch, and at least one light emitting diode, wherein the power supply unit, the switch, and the light emitting diode coupled to the circuit board and electrically connected to each other. Therefore, it is more convenient to carry the phototherapy device due to the volume of the phototherapy device is reduced, and reduces cost effectively. Also, the phototherapy device adopts the SMD (Surface Mount Device) design to be suitable for attaching on the skin, and provides more choice to the consumers.

### FIELD OF THE INVENTION

The present invention relates to a phototherapy device, and in particular to a phototherapy device that features patch design to be suitable for sticking on the skin, and provides more choice to the consumers.

### BACKGROUND OF THE INVENTION

The generalized phototherapy contains the monochromatic wavelength laser and the multi-chromatic wavelength Intense Pulsed Light. The different wavelength of radiation and the different energy may cause the skin to have the different treatment result. Take the laser as the example, its can release the energy in the extremely short time. The laser is a monochromatic wavelength radiation and may cause the selective destruction, in which apply to epilating, spot treatment, dermabrasion, mole removal, and etc. Moreover, take the Intense Pulsed Light as the example, it combines several laser to form a multi-chromatic light and high energy pulsed radiation to alive the skin cell by the non-invasive treatment, and treat variety skin problem such as pigment spots, vessel dilatation, skin pores expansion, wrinkles, and floppy skin. However, the volume of said phototherapy device is huge usually for more storage and costs. The consumers have to pay more to treat said problems of their skin in a medical & aesthetic institution. Therefore, it is necessary to improve the phototherapy device to provide the characteristic of reducing cost, privacy protection, and storing up easily.

### SUMMARY OF THE INVENTION

An object of the present invention is to provide a phototherapy device to reduce the volume and cost of the phototherapy device effectively. Also, the phototherapy device adopts the SMD (Surface Mount Device) design to be suitable for attaching on the skin, and provides more choice to the consumers.

To realize the above objects, the present invention provides a phototherapy device comprises a translucent insulating carrier, and an illuminant module disposed inside the translucent insulating carrier, wherein the illuminant module comprises a circuit board, a power supply unit, a switch, and at least one light emitting diode, wherein the power supply unit, the switch, and the light emitting diode coupled to the circuit board and electrically connected to each other.

In the above-discussed phototherapy device, the translucent insulating carrier is a patch.

In the above-discussed phototherapy device, the translucent insulating carrier was made of translucent plastic.

In the above-discussed phototherapy device, the first elastic yarns and the second elastic yarns are each spandex yarn.

In the above-discussed phototherapy device, the translucent insulating carrier further includes an adhesive layer which disposed on a surface of the translucent insulating carrier.

### BRIEF DESCRIPTION OF THE DRAWINGS

The present invention will be apparent to those skilled in the art by reading the following description of preferred embodiments thereof with reference to the drawings, in which:

FIG. 1 is a upper side view showing a phototherapy device according to the present invention;

FIG. 2 is a bottom side view showing a phototherapy device according to the present invention;

FIG. 3 is a cross-sectional view showing a phototherapy device according to the present invention.

### DETAILED DESCRIPTION OF THE PREFERRED EMBODIMENTS

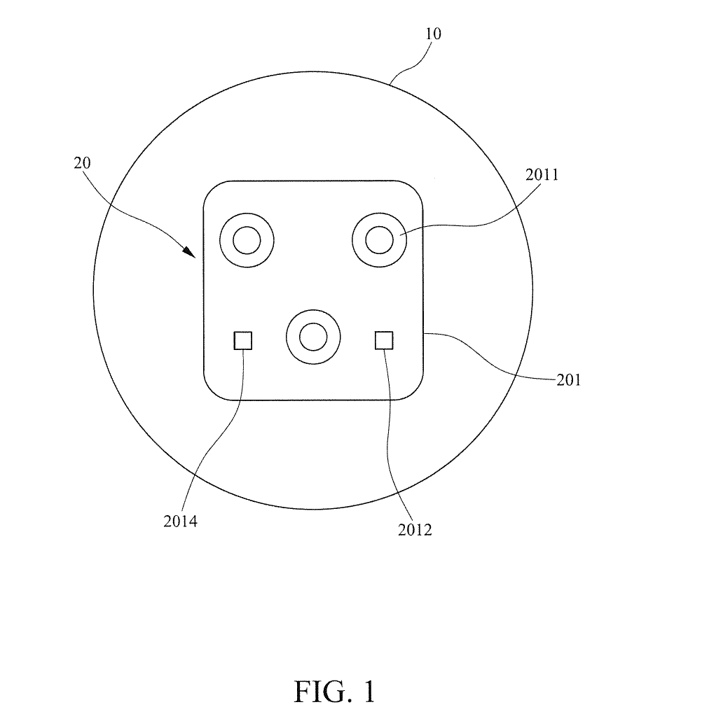

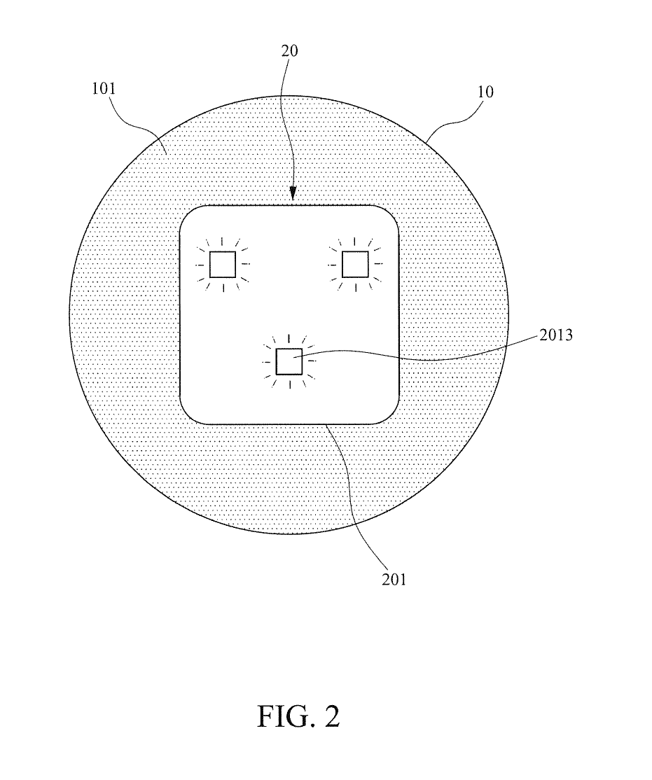

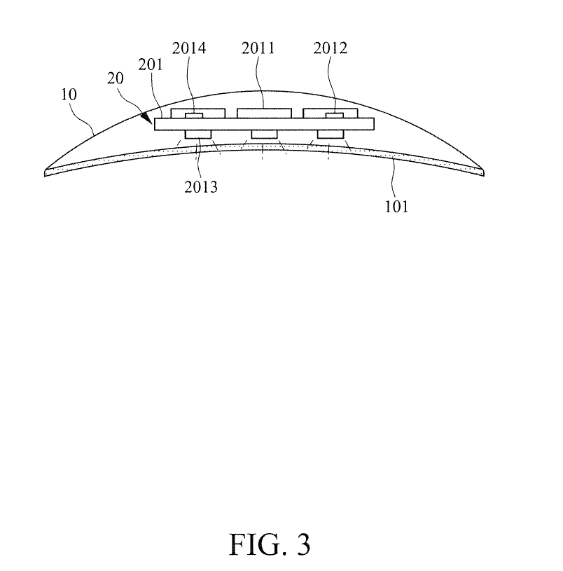

With reference to the drawings and in particular to FIG. 1 to FIG. 3, which is showing a phototherapy device according to the present invention, in the instant embodiment, the phototherapy device according to the present invention comprises a translucent insulating carrier 10, and an illuminant module 20 disposed inside the translucent insulating carrier 10, wherein the illuminant module 20 comprises a circuit board 201, a driver 2014, a power supply unit 2011, a switch 2012, and at least one light emitting diode 2013, in which the power supply unit 2011, the switch 2012, and the light emitting diode 2013 coupled to the circuit board and electrically connected to each other. In the instant embodiment, the translucent insulating carrier 10 is a patch made of silicone, rubber, PI, PU, or PS etc, and the translucent insulating carrier 10 further includes an adhesive layer 101 disposed on a surface of the translucent insulating carrier 10.

With reference to the drawings and in particular to FIG. 1 to FIG. 3, which is showing a phototherapy device according to the present invention, in the instant embodiment, the adhesive layer 101 of the translucent insulating carrier 10 attached to the position of a patient which need a treatment. The translucent insulating carrier 10 is convenient to use to provide better privacy and increase the consumer's willingness to use. When the translucent insulating carrier 10 is attaching on a human body and after turn on the switch 2012 to electrify, the driver 2014 alight the light emitting diode 2013. (In the instant embodiment, the wavelength of the light emitting diode 2013 is 390-470 nm, 500-550 nm, 580-600 nm, 620-670 nm, or 700-1000 nm etc.) The power supply unit 2011 supply the power ceaselessly to alight the light emitting diode 2013. To turn off the light emitting diode 2013, press the switch 2012 to switch off the power supply 2011. Thus, the operation is simple and convenient for the consumers, and the production of SMD designed provides more effective in reducing costs.

According to the phototherapy device described above, the volume and costs of the phototherapy device was reduced by the arrangement of the translucent insulating carrier 10 and the illuminant module 20. Also, the phototherapy device adopts the SMD design to be suitable for attaching on the skin, and provides more choice to the consumers.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A phototherapy device, comprising:
a translucent insulating carrier; and
an illuminant module disposed inside the translucent insulating carrier, comprises a circuit board, a power supply unit, a switch, and at least one light emitting diode, wherein the power supply unit, the switch, and the light emitting diode coupled to the circuit board and electrically connected to each other.

2. The phototherapy device as claimed in claim 1, wherein the translucent insulating carrier is a patch.

3. The phototherapy device as claimed in claim 1, wherein the translucent insulating carrier was made of translucent plastic.

4. The phototherapy device as claimed in claim 1, wherein the translucent insulating carrier further includes an adhesive layer which disposed on a surface of the translucent insulating carrier.
